(19) United States
(12) Patent Application Publication
(10) Pub. No.: US 2023/0194516 A1
(43) Pub. Date: Jun. 22, 2023

(54) METHOD FOR IMMOBILIZING NUCLEIC ACID COMPOUND, REAGENT KIT, AND SENSOR

(71) Applicant: KABUSHIKI KAISHA TOSHIBA, Tokyo (JP)

(72) Inventors: Hiroko MIKI, Kawasaki Kanagawa (JP); Yoshiaki SUGIZAKI, Fujisawa Kanagawa (JP); Hideyuki TOMIZAWA, Ota Gumma (JP)

(73) Assignee: KABUSHIKI KAISHA TOSHIBA, Tokyo (JP)

(21) Appl. No.: 17/930,412

(22) Filed: Sep. 7, 2022

(30) Foreign Application Priority Data
Dec. 16, 2021 (JP) 2021-204337

(51) Int. Cl. G01N 33/543; B82Y 15/00; B82Y 30/00

(52) U.S. Cl. CPC G01N 33/54353; B82Y 15/00; B82Y 30/00

(57) ABSTRACT

According to one embodiment, a method for immobilizing a nucleic acid compound on a surface of a sensor element including graphene, graphene oxide, a carbon nanotube, or graphite, the method includes preparing an aqueous solution containing a nucleic acid compound and sodium chloride, wherein the nucleic acid compound includes a polycyclic aromatic moiety including a polycyclic aromatic skeleton and a linker structure bonded to the polycyclic aromatic skeleton, and a nucleic acid moiety bonded to the linker structure, and dropping the aqueous solution onto the surface of the sensor element.

### CROSS REFERENCE TO RELATED APPLICATIONS

This application is based upon and claims the benefit of priority from Japanese Patent Application No. 2021-204337, filed Dec. 16, 2021, the entire contents of which are incorporated herein by reference.

### FIELD

Embodiments described herein relate generally to a method for immobilizing a nucleic acid compound, a reagent kit, and a sensor.

### BACKGROUND

In order to increase the sensitivity of a sensor, it is required to highly efficiently immobilize an aptamer on a sensor by a simpler method to form a probe at a high density.

### BRIEF DESCRIPTION OF THE DRAWINGS

FIG. 1 is a flowchart showing an example of a method for immobilizing a nucleic acid compound according to a first embodiment.

FIG. 2 is a flowchart showing an example of the method for immobilizing a nucleic acid compound according to the first embodiment.

FIG. 3 is a schematic diagram showing an example of a sensor according to a third embodiment.

FIG. 4 is a plurality of schematic cross-sectional views showing an example of a sensor element portion of the sensor according to the third embodiment, in which part (a) of FIG. 4 shows a state before the sensor according to the third embodiment is subjected to use, and part (b) of FIG. 4 shows a state after the sensor according to the third embodiment is subjected to use.

FIG. 5 is a schematic diagram showing an example of the sensor according to the third embodiment.

FIG. 6 is a graph showing experimental results of Example 1.

### DETAILED DESCRIPTION

In general, according to one embodiment, a method for immobilizing a nucleic acid compound more simply and at a high density, a reagent kit, and a sensor configured to be subjected to implementation of the method or use of the reagent kit are provided.

Hereinafter, various embodiments will be described with reference to the drawings. Each drawing is a schematic diagram for promoting the embodiments and understanding thereof, and its shapes, dimensions, comparisons, and the like are different from actual ones, but these can be modified in design as appropriate in consideration of the following descriptions and known techniques.

### First Embodiment

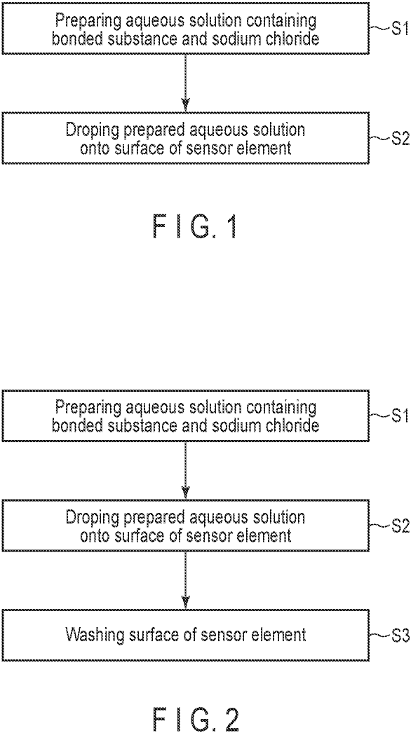

A method for immobilizing a nucleic acid compound according to an embodiment is a method for immobilizing a nucleic acid compound on a surface of a sensor element including graphene, graphene oxide, a carbon nanotube, or graphite, and as shown in FIG. 1, the method includes:
(S1) preparing an aqueous solution containing a nucleic acid compound and sodium chloride; and
(S2) dropping the prepared aqueous solution onto the surface of the sensor element,
   wherein the nucleic acid compound includes a polycyclic aromatic moiety including a polycyclic
aromatic skeleton having an affinity for the surface of the sensor element and a linker structure bonded to the polycyclic aromatic skeleton, and a nucleic acid moiety bonded to the linker structure of the polycyclic aromatic moiety.

The polycyclic aromatic skeleton refers to a molecule of an aromatic compound having two or more cyclic structures in the molecule and a derivative thereof. The polycyclic aromatic skeleton may be a structure having a fused ring, for example, acenes such as naphthalene and anthracene, phenanthrene, and pyrene, or may be a structure having two or more rings, for example, biphenyl, terphenyl, and triphenylmethane separately. The polycyclic aromatic skeleton may be, for example, a molecule of a heterocyclic compound such as quinoline or coumarin. The polycyclic aromatic skeleton may be, for example, a molecule of a nonbenzenoid aromatic compound such as azulene.

In the polycyclic aromatic skeleton exemplified above, since n electrons are delocalized in a cyclic manner to form a thermodynamically stable ring system, n-n interaction occurs between graphene, graphene oxide, a carbon nanotube, and graphite in which n electrons are also delocalized in a cyclic manner. Therefore, the polycyclic aromatic skeleton is easily adsorbed to graphene, graphene oxide, a carbon nanotube, and graphite, that is, has an affinity for graphene, graphene oxide, a carbon nanotube, and graphite.

The polycyclic aromatic moiety according to the method for immobilizing a nucleic acid compound of the embodiment refers to a compound including the above-mentioned polycyclic aromatic skeleton and a linker structure bonded to the polycyclic aromatic skeleton. The linker structure suppresses steric interference between the nucleic acid and the surface of the sensor element caused by directly bonding the nucleic acid to the aromatic ring of the polycyclic aromatic skeleton. The nucleic acid compound may not include the linker structure as long as the adsorption property of the polycyclic aromatic skeleton to the surface of the sensor element is maintained. The linker structure includes at least two carbons between the bonding position to the polycyclic aromatic skeleton and the bonding position to the nucleic acid moiety. For example, the linker structure includes at least one carbon-carbon single bond between the bonding position to the polycyclic aromatic skeleton and the bonding position to the nucleic acid moiety. At least one carbon constituting the linker structure, for example, the carbon closest to the bonding position to the nucleic acid moiety or the carbon bonded to the nucleic acid moiety is not included in the plane formed by the carbons constituting the polycyclic aromatic skeleton. The terminal of the linker structure is preferably a hydrophilic group which is easily bonded to the nucleic acid moiety, and when the linker structure has a phosphate group as a hydrophilic group, the nucleic acid moiety can be synthesized by adding a nucleotide starting from the phosphate group. The terminal of the linker structure can be bonded to, for example, the 5′ end or 3′ end of the nucleic acid moiety via a phosphate bond, a phosphoester bond, or the like.

The linker structure bonded to the polycyclic aromatic skeleton is, for example, a linker structure in which the carbon at the 5-position of the pyrimidine ring of deoxyuridine is alkynylated (the following formula (1)) or a linker structure in which a phosphate group is bonded to the carbon at the 2-position of the pyrrolidine ring (the following formula (2)).

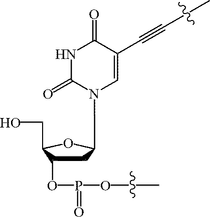

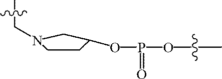

The nucleic acid constituting the nucleic acid moiety may be a single-stranded nucleic acid, is not limited to a DNA or RNA molecule, and may be various artificial nucleic acids such as GNA, LNA, PNA, and TNA. The nucleic acid moiety may be an aptamer which binds to a particular substance. In addition, the nucleic acid moiety may be optionally modified, and any protecting group may be introduced. The base length of the nucleic acid moiety is not particularly limited, and can be several bases to several hundred bases.

The nucleic acid compound according to the method for immobilizing a nucleic acid compound of the embodiment is a compound formed by bonding the polycyclic aromatic moiety and the nucleic acid moiety mentioned above, at the terminal of the linker structure bonded to the polycyclic aromatic skeleton constituting the polycyclic aromatic moiety. For example, when the polycyclic aromatic skeleton is pyrene, the linker structure is the structure represented by the above formula (1), and the nucleic acid moiety is DNA, the nucleic acid compound is a compound represented by the following formula (3).

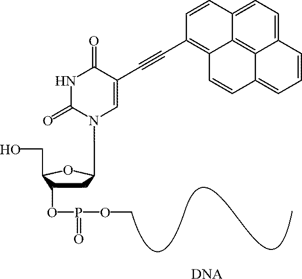

In addition, for example, when the polycyclic aromatic skeleton is pyrene, the linker structure is the structure represented by the above formula (2), and the nucleic acid moiety is DNA, the nucleic acid compound is a compound represented by the following formula (4).

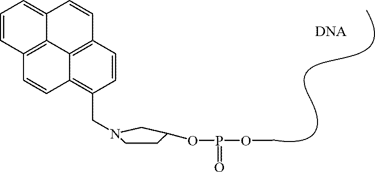

Since the nucleic acid compound includes the polycyclic aromatic skeleton mentioned above, the nucleic acid compound is easily adsorbed to graphene, graphene oxide, a carbon nanotube, and graphite, and has an affinity for graphene, graphene oxide, a carbon nanotube, and graphite. Therefore, in the method for immobilizing a nucleic acid compound according to the embodiment, by dropping the solution containing the nucleic acid compound onto the surface of the sensor element including graphene, graphene oxide, a carbon nanotube, or graphite, the nucleic acid compound and thus the nucleic acid moiety constituting the nucleic acid compound can be immobilized on the surface of the sensor element.

The solvent of the solution containing the nucleic acid compound used in the method for immobilizing a nucleic acid compound according to the embodiment is water, and sodium chloride is contained as a solute other than the nucleic acid compound. In other words, the solution containing the nucleic acid compound is an aqueous sodium chloride solution. As will be mentioned later, since sodium chloride has an action of promoting the immobilization of the nucleic acid compound on the surface of the sensor element, the immobilization of the nucleic acid moiety can be promoted as the added amount of sodium chloride is increased. Therefore, the concentration of sodium chloride in the solution containing the nucleic acid compound is preferably higher, for example, 150 mM or more.

The solution containing the nucleic acid compound may contain any solute, but the concentration of a phosphate ion and 2-[4-(2-hydroxyethyl)-1-piperazinyl]-ethanesulfonic acid (hereinafter referred to as “HEPES”) is preferably lower, and it is more preferable that a phosphate ion and HEPES are not contained in the solution. This is because, as will be mentioned later, a phosphate ion and HEPES have an action of inhibiting the immobilization of the nucleic acid compound on the surface of the sensor element.

Conventionally, a method for immobilizing a nucleic acid compound on a surface of a sensor element including, for example, graphene is performed by the following steps (a) to (d):
(a) dropping an organic solvent containing 1-pyrenebutanoic acid and a succinimide ester onto the surface of the sensor element;
(b) after step (a), washing the surface of the sensor element;
(c) after step (b), dropping a buffer containing NH₂-DNA onto the surface of the sensor element; and
(d) after step (c), adding an ethanolamine solution onto the surface of the sensor element.

Here, in the above step (a), by bonding 1-pyrenebutanoic acid to the surface of the sensor element, and by bonding a succinimide ester to the 1-pyrenebutanoic acid bonded to the surface of the sensor element, a scaffold molecule (pyrene derivative) of the nucleic acid molecule is immobilized on the surface of the sensor element.

In the above step (b), 1-pyrenebutanoic acid and a succinimide ester which have not been immobilized as the scaffold molecule of the nucleic acid molecule are removed from the surface of the sensor element.

In the above step (c), by bonding the scaffold molecule immobilized on the surface of the sensor element through the above step (a) to NH₂-DNA as the nucleic acid molecule, the nucleic acid molecule is immobilized on the surface of the sensor element.

In the above step (d), a scaffold molecule not bonded to the nucleic acid molecule is inactivated, and the surface of the sensor element is washed.

As mentioned above, the conventional method for immobilizing a nucleic acid probe has a problem in which the total number of steps is large and the method is complicated. Furthermore, there is also a problem in which graphene may be peeled off from the surface of the sensor element by the organic solvent used in step (a).

On the other hand, in the method for immobilizing a nucleic acid compound according to the embodiment, the nucleic acid compound can be immobilized on the surface of the sensor element only by dropping the aqueous solution containing the nucleic acid compound and sodium chloride onto the surface of the sensor element.

Since the nucleic acid compound is a compound in which the polycyclic aromatic skeleton, the linker structure, and the nucleic acid moiety are bonded, the method for immobilizing a nucleic acid compound according to the embodiment does not require a step of bonding the polycyclic aromatic skeleton and the linker structure on the surface of the sensor element (namely, step (a) of the conventional method for immobilizing a nucleic acid compound). Furthermore, since step (b) and step (d) of the conventional method for immobilizing a nucleic acid compound are steps required in association with step (a), the method for immobilizing a nucleic acid compound according to the embodiment also does not require steps corresponding to step (b) and step (d) of the conventional method for immobilizing a nucleic acid compound. Therefore, the method for immobilizing a nucleic acid compound according to the embodiment has fewer steps than those of the conventional method for immobilizing a nucleic acid compound, and can immobilize a nucleic acid compound more simply.

Furthermore, since the method for immobilizing a nucleic acid compound according to the embodiment does not require an organic solvent, there is no possibility that graphene is peeled off from the surface of the sensor element by the organic solvent, which is preferable.

In a further embodiment, as shown in FIG. 2, step (S3) of washing the surface of the sensor element may be performed after step (S2). An object of step (S3) is to remove the nucleic acid compound or the like remaining in the solution without being immobilized on the surface of the sensor element. The surface of the sensor element may be washed by, for example, replacing with an aqueous solution containing no nucleic acid compound.

### Second Embodiment

A reagent kit according to a second embodiment is a reagent kit used for immobilizing a nucleic acid compound on a surface of a sensor element including graphene, graphene oxide, a carbon nanotube, or graphite.

The reagent kit according to the second embodiment includes a first container which accommodates a nucleic acid compound including a polycyclic aromatic moiety including a polycyclic aromatic skeleton having an affinity for the surface of the sensor element and a linker structure bonded to the polycyclic aromatic skeleton, and a nucleic acid moiety bonded to the linker structure of the polycyclic aromatic moiety, and a second container which accommodates an aqueous sodium chloride solution.

The nucleic acid compound of the reagent kit according to the second embodiment is the same as the nucleic acid compound in the method for immobilizing a nucleic acid compound according to the first embodiment. The nucleic acid compound accommodated in the first container is more stable when the nucleic acid compound is in a solid state and dried. Therefore, it is preferable that the first container is configured such that the accommodated nucleic acid compound is stored in a moisture-proof manner.

When the reagent kit according to the second embodiment is used, a composition accommodated in the first container is weighed, and by adding the aqueous sodium chloride solution stored in the second container according to the amount of the composition or by adding a predetermined amount of the aqueous sodium chloride solution stored in the second container to the first container, an aqueous solution containing the composition and sodium chloride is prepared.

By dropping the aqueous solution containing the composition and sodium chloride prepared as mentioned above onto the surface of the sensor element, the polycyclic aromatic skeleton constituting the nucleic acid compound is adsorbed on the surface of the sensor element, and thus the nucleic acid moiety can be immobilized on the surface of the sensor element.

In addition, as a further embodiment, the nucleic acid compound may be stored in a state of being dissolved in a liquid in the first container, and further, a stabilizer for the nucleic acid compound may be contained in the liquid.

### Third Embodiment

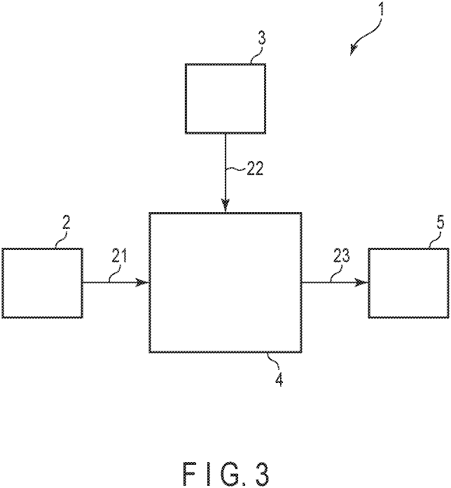

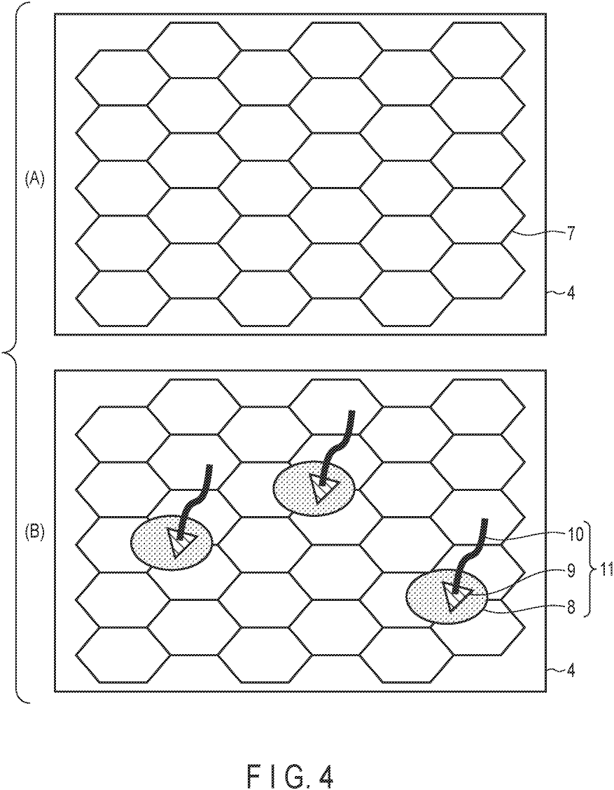

A sensor according to a third embodiment is a sensor configured to be subjected to implementation of the method according to the first embodiment or use of the reagent kit according to the second embodiment. Hereinafter, a structure of the sensor according to the third embodiment will be described in detail with reference to FIGS. 3 and 4.

As shown in FIG. 3, a sensor 1 comprises a first container 2 configured to accommodate an aqueous solution (first solution) containing a nucleic acid compound and sodium chloride, a second container 3 configured to accommodate a measurement solution (second solution), a sensor element 4 including graphene, graphene oxide, a carbon nanotube, or graphite, and a third container 5 configured to accommodate a liquid discharged from a surface of the sensor element 4. The nucleic acid compound in the sensor according to the third embodiment is the same as the nucleic acid compound described in the first embodiment and the second embodiment.

The second solution is, for example, a solution containing at least any one of an ionic liquid for enhancing the measurement sensitivity of the sensor 1, a buffer for enhancing the stability of the sensor 1, and a surfactant/chelating agent for enhancing the stability of a nucleic acid moiety. The second solution can contain, as the ionic liquid, any ionic liquid such as choline dihydrogen phosphate, an imidazolium salt-based ionic liquid, a pyrrolidinium salt-based ionic liquid, a pyridinium salt-based ionic liquid, a piperidinium salt-based ionic liquid, an ammonium salt-based ionic liquid, a phosphonium salt-based ionic liquid, or a phosphonate-based ionic liquid. The second solution can contain, as the buffer, any buffer such as a phosphate buffer or an HEPES buffer. The second solution can contain, as the chelating agent, an aminocarboxylate such as EDTA.

FIG. 4 shows an example of a sensor element portion of the sensing device of the third embodiment. As shown in part (a) of FIG. 4, before the sensor according to the third embodiment is subjected to use, that is, before the first solution and the second solution are supplied to the surface of the sensor element 4, the nucleic acid compound is not immobilized on the surface of the sensor element 4.

When the sensor according to the third embodiment is subjected to use, the first solution is supplied to the surface of the sensor element 4 via a first flow path 21 extending from the first container 2. When the first solution is supplied, the nucleic acid compound 11 is immobilized on the surface of the sensor element 4 as shown in part (b) of FIG. 4. The nucleic acid compound not bonded to the surface of the sensor element 4 is discharged simultaneously with supply of the second solution.

The sensor 1 according to the third embodiment in which the nucleic acid compound 11 is immobilized on the surface of the sensor element 4 as shown in part (a) of FIG. 4 can be used, for example, as a sensor for use in capturing and measuring a specific target substance, and the nucleic acid moiety 10 of the nucleic acid compound 11 can be used as a probe which specifically binds to and captures a target substance in such a sensor.

When the sensor 1 according to the third embodiment is used as a sensor for capturing and measuring a specific target substance, it is preferable to immobilize the nucleic acid compound 11 on the surface of the sensor element 4 and then replace the first solution on the sensor element 4 with the second solution. This replacement is performed by discharging the first solution on the sensor element 4 via a third flow path 23 and supplying the second solution onto the sensor element 4 via a second flow path 22. By replacing the first solution on the sensor element 4 with the second solution, when the nucleic acid compound not adsorbed to the sensor element 4 remains in the first solution, it is possible to prevent the compound and a target substance from binding to each other to reduce the measurement sensitivity.

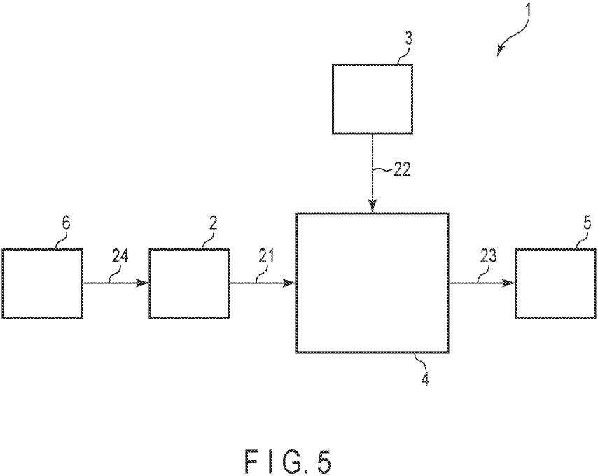

Furthermore, in a further embodiment, the sensor further includes a fourth container 6 which accommodates an aqueous sodium chloride solution, and the first container 2 accommodates the nucleic acid compound in a solid state instead of the first solution. In this case, as shown in FIG. 5, the first container 2 and the fourth container 6 are connected by a fourth flow path 24, and when the sensor is subjected to use, the aqueous sodium chloride solution is supplied from the fourth container 6 to the first container 2 through the fourth flow path 24. In the first container 2 supplied with the aqueous sodium chloride solution, the nucleic acid compound is dissolved in the aqueous sodium chloride solution to prepare the first solution. The first solution prepared in the first container is supplied onto the surface of the sensor element 4 through the first flow path 21.

### EXAMPLES

Hereinafter, experiments performed using the method for immobilizing a nucleic acid compound according to the embodiment will be described.

### Example 1. Comparison of Immobilized Amounts of Nucleic Acid Compound Under Different Solutes

Nine types of aqueous solutions having different solutes and concentrations (aqueous solutions A to I) were prepared. The respective compositions are shown in Table 1 below.

Here, aqueous solutions A to H commonly contain a nucleic acid compound represented by the following formula (5) at a concentration of 1 µM as a solute as shown in Table 1. Aqueous solution I is pure water and does not contain a solute. In the table, “D-PBS(-)” refers to a buffer containing KCl at a concentration of 200 mg/L, NaCl at a concentration of 8,000 mg/L, KH₂PO₄ at a concentration of 200 mg/L, and Na₂HPO₄ at a concentration of 1,150 mg/L, and “PB” refers to a phosphate buffer.

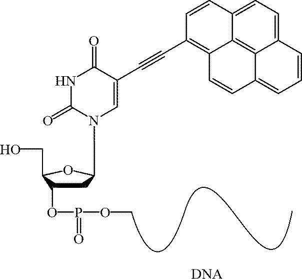

Here, the DNA in the above formula (5) is 40-base single-stranded DNA.

Respective aqueous solutions A to I were dropped onto different graphene surfaces. After a lapse of 60 minutes from the dropping, each graphene surface was analyzed by X-ray photoelectron spectroscopy (XPS) analysis. The conditions for the XPS analysis were as follows: excited X-ray: monochromatic Al Kα₁_{,}₂ ray (1,486.6 eV), X-ray diameter: 100 µm, photoelectron detection angle: 45° (inclination of the detector with respect to the sample surface).

The atomic ratio (N/Si or P/Si) was calculated from the elemental composition (atomic%), and the P/Si value was used as an index of the immobilized amount. The P/Si value in NaCl (150 mM) as the solute was defined as 1, and the relative ratio of the immobilized amount of the nucleic acid compound when each solute was contained was calculated.

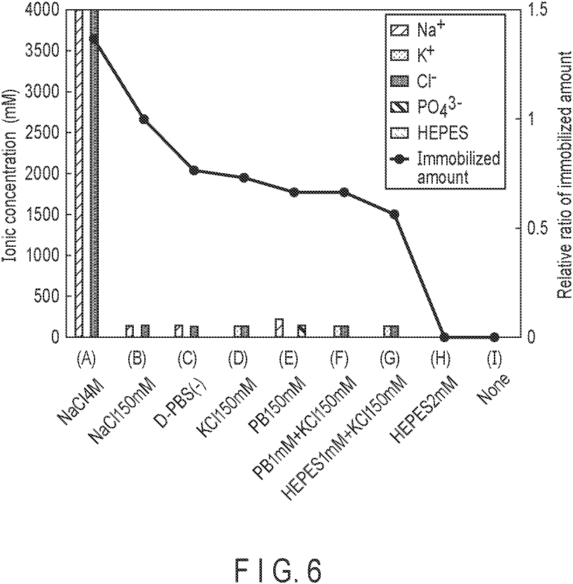

The calculation results of the relative ratio of the immobilized amount of the nucleic acid compound are shown in FIG. 6. Referring to FIG. 6, it can be seen that the immobilized amount in the immobilization method using aqueous solutions A and B is higher than that in the immobilization method using aqueous solutions C to I. Therefore, it was shown that sodium chloride exhibits a higher immobilized amount of the nucleic acid compound than that of other solutes.

Further, referring to FIG. 6, it can be seen that the immobilized amount when aqueous solution A having a composition close to that of an aqueous saturated solution of NaCl was used is higher than the immobilized amount when aqueous solution B was used. This showed that a higher concentration of sodium chloride tended to increase the immobilized amount of the nucleic acid compound, and sodium chloride had an action of promoting the immobilization of the nucleic acid compound.

Furthermore, referring to FIG. 6, it can be seen that the immobilized amount in the immobilization method using aqueous solution F or aqueous solution G is lower than that in the immobilization method using aqueous solution D. Furthermore, it can be seen that the immobilization of the nucleic acid compound was hardly observed when aqueous solution H was used. Therefore, since use of PB or HEPES tends to reduce the immobilized amount of the nucleic acid compound, it was inferred that a phosphate ion and HEPES may have an action of inhibiting the immobilization of the nucleic acid compound.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method for immobilizing a nucleic acid compound on a surface of a sensor element including graphene, graphene oxide, a carbon nanotube, or graphite, the method comprising:
preparing an aqueous solution containing a nucleic acid compound and sodium chloride, wherein the nucleic acid compound includes a polycyclic aromatic moiety including a polycyclic aromatic skeleton and a linker structure bonded to the polycyclic aromatic skeleton, and a nucleic acid moiety bonded to the linker structure; and
dropping the aqueous solution onto the surface of the sensor element.

2. The method according to claim 1, wherein the polycyclic aromatic skeleton is pyrene.

3. The method according to claim 1, wherein the linker structure of the polycyclic aromatic moiety comprises a phosphate group at a terminal, and the nucleic acid compound comprises the nucleic acid moiety and the linker structure bonded to each other via the phosphate group.

4. The method according to claim 3, wherein the linker structure of the polycyclic aromatic moiety is a linker structure represented by the following formula (1) or the following formula (2). 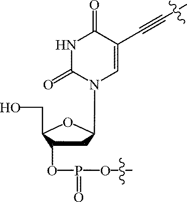 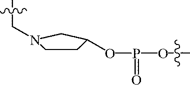
.

5. The method according to claim 4, wherein the nucleic acid compound is a compound represented by the following formula (3) or the following formula (4). 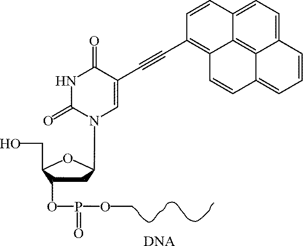 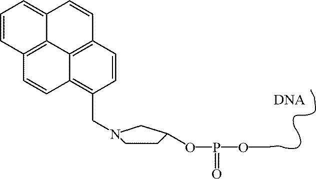
.

6. The method according to claim 1, wherein the aqueous solution does not contain an organic solvent.

7. The method according to claim 1, wherein the aqueous solution contains a sodium chloride at concentration of 150 mM or more.

8. The method according to claim 1, wherein the aqueous solution does not contain a phosphate ion or 2-[4-(2-hydroxyethyl)-1-piperazinyl]-ethanesulfonic acid.

9. The method according to claim 1, further including washing the surface of the sensor element by, onto the surface of the sensor element onto which the aqueous solution has been dropped, dropping an aqueous sodium chloride solution and replacing with the aqueous sodium chloride solution.

10. A reagent kit used for immobilizing a nucleic acid compound on a surface of a sensor element including graphene, graphene oxide, a carbon nanotube, or graphite, the reagent kit comprising:
a first container which accommodates a nucleic acid compound including a polycyclic aromatic moiety including a polycyclic aromatic skeleton and a linker structure bonded to the polycyclic aromatic skeleton, and a nucleic acid moiety bonded to the linker structure of the polycyclic aromatic moiety; and
a second container which accommodates an aqueous sodium chloride solution.

11. The reagent kit according to claim 10, wherein the nucleic acid moiety is DNA or RNA.

12. The reagent kit according to claim 10, wherein the polycyclic aromatic skeleton is pyrene or a derivative of the pyrene.

13. The reagent kit according to claim 10, wherein the linker structure of the polycyclic aromatic moiety is a linker structure represented by the following formula (5) or the following formula (6). 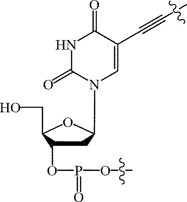 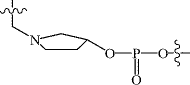
.

14. The reagent kit according to claim 10, wherein the nucleic acid compound is a compound represented by the following formula (7) or the following formula (8). 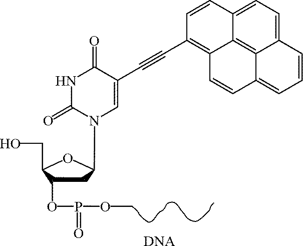 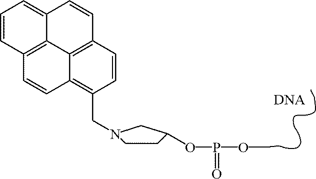
.

15. The reagent kit according to claim 10, wherein the aqueous sodium chloride solution comprises a concentration of 150 mM or more.

16. The reagent kit according to claim 10, wherein the aqueous sodium chloride solution does not comprise a phosphate ion or 2-[4-(2-hydroxyethyl)-1-piperazinyl]-ethanesulfonic acid.

17. The reagent kit according to claim 10, wherein the aqueous sodium chloride solution does not comprise an organic solvent.

18. A sensor configured to be subjected to implementation of the method according to claim 1 or use of the reagent kit according to claim 10, the sensor comprising:
a sensor element including graphene, graphene oxide, a carbon nanotube, or graphite;
a first container which accommodates a first solution;
a second container which accommodates a second solution;
a first flow path configured to supply the first solution from the first container to a surface of the sensor element;
a second flow path configured to supply the second solution from the second container to the surface of the sensor element; and
a third flow path configured to discharge a liquid from the surface of the sensor element,
wherein the first solution is an aqueous solution containing a nucleic acid compound and sodium chloride, wherein the nucleic acid compound includes a polycyclic aromatic moiety including a polycyclic aromatic skeleton and a linker structure bonded to the polycyclic aromatic skeleton, and a nucleic acid moiety bonded to the linker structure of the polycyclic aromatic moiety, and
the second solution is an aqueous solution containing at least any one of a buffer, an ionic liquid, a surfactant, and a chelating agent.

19. A sensor configured to be subjected to implementation of the method according to claim 1 or use of the reagent kit according to claim 10, the sensor comprising:
a sensor element including graphene, graphene oxide, a carbon nanotube, or graphite;
a first container which accommodates a composition of a nucleic acid compound and sodium chloride, wherein the nucleic acid compound includes a polycyclic aromatic moiety including a polycyclic aromatic skeleton and a linker structure bonded to the polycyclic aromatic skeleton, and a nucleic acid moiety bonded to the linker structure of the polycyclic aromatic moiety;
a second container which accommodates a second solution containing at least any one of a buffer, an ionic liquid, a surfactant, and a chelating agent;
a third container configured to accommodate a liquid discharged from the sensor element; and
a fourth container which accommodates an aqueous sodium chloride solution,
wherein the fourth container and the first container are connected by a fourth flow path configured to supply the aqueous sodium chloride solution to the first container,
the first container and the sensor element are connected by a first flow path configured to supply a first solution to a surface of the sensor element, the first solution being produced in the first container by dissolving the nucleic acid compound in the aqueous sodium chloride solution supplied by the fourth flow path,
the second container and the sensor element are connected by a second flow path configured to supply the second solution from the second container to the surface of the sensor element, and
the sensor element and the third container are connected by a third flow path configured to discharge a liquid from the surface of the sensor element.
